(19) United States
(12) Patent Application Publication
(10) Pub. No.: US 2012/0095291 A1
(43) Pub. Date: Apr. 19, 2012

(54) ENDOSCOPE OVERTUBE

(76) Inventor: Kiyokazu Nakajima, Osaka (JP)

(21) Appl. No.: 13/382,159

(22) PCT Filed: Jul. 6, 2010
(86) PCT No.: PCT/JP2010/061472

§ 371 Date: Jan. 4, 2012

(30) Foreign Application Priority Data
Jul. 6, 2009 (JP) 2009-160088

(51) Int. Cl. A61B 1/00

(52) U.S. Cl. 600/121

(57) ABSTRACT

An object of the present invention is to provide means capable of steering a treatment instrument together with an endoscope with good operability in endoscopic surgery. The endoscope overtube of the present invention includes a first insertion passage into which an endoscope is inserted, wherein a wall constituting the first insertion passage includes a second insertion passage into which a treatment instrument is inserted, and a major axis direction of the second insertion passage is different from a major axis direction of the first insertion passage at a distal end of the second insertion passage. Since the treatment instrument insertion passage of the overtube does not extend in a direction along the major axis of the overtube, the treatment instrument is protruded from the distal end of the overtube in a direction different from that of the center of a surgical field, and extends outward, away from a direction along the viewing axis of the overtube. For example, when a grasping forceps is inserted into the insertion passage of the overtube, by adjusting the extent of protrusion of the grasping forceps, it is possible to grip a tissue under an appropriate tension and perform a treatment such as removal with a treatment instrument such as an electrocautery inserted from the treatment instrument channel of the endoscope.

### TECHNICAL FIELD

The present invention relates to an endoscope overtube. More particularly, the invention relates to an overtube that enables a treatment instrument to be protruded in a direction different from the viewing axis of an endoscope with good operability.

### BACKGROUND ART

In general, an endoscope is provided, at its tip portion, with an observation optical system, an illumination optical system for illuminating an affected area, an air supply and water supply channel, a treatment instrument channel, and so forth. By introducing a treatment instrument such as a forceps from the treatment instrument channel while using an endoscope to observe an affected area in a lumen such as stomach from a video camera portion, it is possible to perform various maneuvers such as specimen retrieval, removal of a foreign body, hemostasis, resection of a tumor, and gallstone. Overtubes for assisting insertion of an endoscope or for simultaneous insertion of a plurality of endoscopes or treatment instruments are also known (for example, Patent Document 1).

In recent years, new medical treatment techniques using an flexible endoscope, including, for example, endoscopic mucosal resection (EMR), endoscopic submucosal dissection (ESD), endoscopic aspiration mucosectomy (EAM), and endoscopic variceal ligation (EVL) have been developed and are receiving attention in the field of digestive system diseases. Recently, clinical introduction of the surgery called Natural Orifice Translumenal Endoscopic Surgery (NOTES) (surgery by which an flexible endoscope is inserted, for example, from mouth or anus into a lumen and is caused to reach the inside of a body cavity by incising the wall of the lumen) has also been started. These medical treatments using a flexible endoscope do not theoretically require incision of a body surface, and are therefore expected to become one of the pillars of medical care in the 21st century as patient friendly, minimally invasive medical treatment techniques.

Such maneuvers using a flexible endoscope and an overtube that are currently available have some technological problems. To perform a treatment endoscopically, it is necessary to insert a long and flexible treatment instrument through an insertion passage (treatment instrument channel) provided in an endoscope or overtube. This channel is provided along the major axis (viewing axis) of the endoscope, and therefore the treatment instrument is also protruded from the tip of the endoscope along the viewing axis. Accordingly, the treatment instrument extends parallel to the viewing axis directly to the center of a surgical field. Consequently, operations that require a sufficient distance between the surgical site and the treatment instrument and operations that require lateral movement rather than movement in the axial direction, including, for example, the operation of applying tension (traction) to a tissue to be treated, cannot be performed easily and appropriately. Also for a multichannel endoscope provided with two or more channels, a plurality of inserted treatment instruments extends parallel to each other to the center of a surgical field, and therefore it is not possible to arrange the treatment instruments at a predetermined distance and a predetermined angle. Accordingly, the technical difficulties of EMR, ESD, NOTES, and the like are very high.

Various investigations have been conducted to determine how to orient a treatment instrument at an angle different from the major axis direction of an endoscope or overtube at the tip of the endoscope or overtube. Mainly, it has been investigated to make the treatment instrument itself deflectable or steerable.

For example, Patent Document 2 discloses a tool arm including a shaft having a proximal end and a deflectable or steerable distal end. This tool arm includes, for example, a plurality of adjacent links, and these links are pivotally attached by hinged structures. Alternatively, the tool arm includes, for example, a pullwire for deflecting the steerable distal end. Thus, a deflectable treatment instrument has a very complex structure.

In another known overtube, a treatment instrument insertion channel is arranged outside an overtube having an endoscope insertion channel, and the overtube includes, in the vicinity of its tip, means for adjusting the relative distance between the endoscope and the treatment instrument (Patent Document 3). Although this overtube guides the direction of the treatment instrument by being curved, the overtube itself has a very complex structure.

### PRIOR ART DOCUMENTS

### Patent Documents

Patent Document 1: Japanese Laid-Open Patent Publication No. 2008-125819

Patent Document 2: Japanese National Publication No. 2007-511247

Patent Document 3: Japanese Laid-Open Patent Publication No. 2000-33071

### SUMMARY OF INVENTION

### Problems to be Solved by the Invention

It is an object of the present invention to provide means capable of steering a treatment instrument together with an endoscope with good operability in endoscopic surgery.

### Means for Solving the Problems

The present inventor accomplished the present invention on the basis of the finding that the direction in which a treatment instrument is protruded to the center of a surgical field and the direction of the viewing axis of an endoscope can be made deliberately different by providing a treatment instrument insertion passage in the form of a helix in the wall of an endoscope overtube.

The present invention provides an endoscope overtube including a first insertion passage into which an endoscope is inserted, wherein a wall constituting the first insertion passage includes a second insertion passage into which a treatment instrument is inserted, and a major axis direction of the second insertion passage is different from a major axis direction of the first insertion passage at a distal end of the second insertion passage.

In one embodiment, the major axis direction of the second insertion passage forms a helix around the major axis direction of the first insertion passage as a central axis, throughout the second insertion passage.

In one embodiment, the helix has a pitch of one turn (360°) or two turns (720°) throughout the second insertion passage.

In one embodiment, the second insertion passage is a lumen that is independent of the first insertion passage.

The present invention further provides a method for using an endoscope overtube, including the step of turning an endoscope overtube around an endoscope.

### Effects of Invention

The endoscope overtube of the present invention is characterized in that (1) a treatment instrument insertion passage is provided in the wall of an already-existing endoscope overtube and that (2) the treatment instrument insertion passage of the overtube extends preferably in a helical direction, rather than a direction along the major axis of the overtube. Accordingly, the treatment instrument is protruded from the distal end of the overtube in a direction different from that of the center of a surgical field, and extends outward, away from a direction along the viewing axis of the overtube. For example, when the treatment instrument inserted into the insertion passage of the overtube is a grasping forceps, the grasping forceps protrudes in a direction outward of the direction of the viewing axis of the overtube. By adjusting the extent of protrusion of the grasping forceps, it is possible to grip a tissue under an appropriate tension and perform a treatment (for example, incision, dissection) with another treatment instrument (for example, an electrocautery, a dissecting forceps) inserted from the treatment instrument channel of the endoscope. Accordingly, with the use of the endoscope overtube of the present invention, it is possible to dramatically improve the operability and the safety of medical treatments that use a flexible endoscope.

### BRIEF DESCRIPTION OF DRAWINGS

FIG. 1 is a side view of an endoscope overtube according to the present invention.

FIG. 2(A) is a perspective view of the distal side of the endoscope overtube of the present invention when an endoscope is inserted into a first insertion passage, and FIG. 2(B) is an enlarged view showing an area in the vicinity of the distal end.

FIG. 3 is a schematic diagram showing the endoscope overtube of the present invention when an endoscope is inserted into the first insertion passage, an electrocautery is inserted into a treatment instrument channel of the endoscope, and a grasping forceps is inserted into a second insertion passage of the overtube, with FIG. 3(A) showing a state in which the electrocautery and the grasping forceps are protruded from the distal end of the overtube and FIG. 3(B) showing a state in which the electrocautery and the grasping forceps are further protruded from the state in FIG. 3(A).

FIG. 4 is a schematic diagram showing a surgical field when endoscopic surgery is performed in a digestive tract by using the endoscope overtube of the present invention.

### MODE FOR CARRYING OUT THE INVENTION

In the present invention, an “endoscope” refers to a medical flexible endoscope unless otherwise stated. Such flexible endoscopes are formed of a flexible material, and can be classified as those using, as the optical system contained therein, glass fiber and those using a CCD. The light source is provided in a control device outside the body, a light source that guides light with optical fiber and irradiates the light from its tip portion is commonly used. Another type of the endoscope contains an LED at its tip. In general, an endoscope includes a path (sub-lumen or channel) different from that of the optical system, and enables local irrigation, injection of a gas and a liquid, spraying of medicine, aspiration, treatments using dedicated treatment instruments (devices), and so forth. In addition, the orientation of the tip of the endoscope can be freely changed by an operation at hand.

The size of the endoscope used in the present invention can be appropriately selected according to the hollow organ of interest. Examples of arbitrary hollow organs of interest include esophagus, stomach, small intestine, colon and rectum, vagina, and bladder.

As used herein, the term “proximal” refers to a portion of an instrument and an apparatus that is closer to the operator, and the term “distal” refers to a portion of the instrument and the apparatus that is farther from the operator.

The endoscope overtube of the present invention includes a first insertion passage into which an endoscope is inserted, wherein a wall constituting the first insertion passage includes a second insertion passage into which a treatment instrument is inserted, and the major axis direction of the second insertion passage is different from the major axis direction of the first insertion passage at a distal end of the second insertion passage.

The material of the overtube is a material commonly used for a medical instrument, and is required to have flexibility, less friction (lubricity), strength, column stiffness, and so forth. Examples of polymers that can be used for such a medical instrument include flexible resins such as polyvinyl chloride, polyethylene, polyester, polyurethane, and polyamide. Polyvinyl chloride is preferably in terms of lesser friction.

Preferably, the endoscope overtube of the present invention is a cylindrical body. For example, when the overtube is intended for use for surgery of stomach and esophagus, it preferably has a form that is curved into an arched shape for facilitating its insertion from the oral region to the esophagus of a subject. There is no particular limitation on the outer diameter of the endoscope overtube of the present invention. The outer diameter may be a size that does not cause an excessive expansion of a lumen into which the overtube is to be inserted, preferably 20 mm or less, more preferably 18 mm or less, even more preferably 15 to 18 mm.

The diameter of the first insertion passage of the endoscope overtube of the present invention (hereinafter, also referred to as the inner diameter of the overtube) may be any size that allows insertion of an endoscope. Since endoscopes having a very small diameter of 5 mm exist, the inner diameter of the overtube is preferably 5 mm or greater.

In the endoscope overtube of the present invention, the wall constituting the first insertion passage includes the second insertion passage, and therefore the overtube may have a thickness than that of conventional overtubes. There is no particular limitation on the wall thickness as long as it is a thickness that allows formation of the second insertion passage for the treatment instrument and it allows formation of the first insertion passage for the endoscope. The wall thickness may be appropriately decided according to the outer diameter and the inner diameter of the overtube as well as the shape and the inner diameter of the second insertion passage, which will be described later. For example, when the outer diameter of the overtube is 18 mm and the inner diameter thereof is 12 mm, the wall thickness of the overtube is 3 mm. The wall thickness is preferably 2 mm or greater, more preferably 3 mm or greater, and preferably 5 mm or less, more preferably 4 mm or less.

The shape and the size of the second insertion passage of the endoscope overtube of the present invention may be appropriately decided in consideration of treatment instruments that are commonly used in the art. Although the second insertion passage of the overtube may have the shape of a groove opened in the first insertion passage, it is preferably a lumen that is independent of the first insertion passage. When the second insertion passage is an independent lumen, the second insertion passage can be appropriately used not only for insertion of the treatment instrument, but also for air supply, water supply, smoke exhaustion, insertion of an auxiliary treatment instrument, insertion of a second endoscope, and so forth. Note that the second insertion passage may also be used for these applications (excluding the water supply function) when it has a groove shape.

The major axis direction of the second insertion passage of the endoscope overtube of the present invention is different from the major axis direction of the first insertion passage at a distal end of the second insertion passage. As used herein, the major axis direction of an insertion passage refers to the direction of the axial center along the longitudinal direction of the insertion passage. The major axis direction of the second insertion passage may be parallel to the major axis direction of the first insertion passage on the proximal end side, but the major axis directions of the first and second insertion passages should not be parallel at their distal ends. The reason is that, when they are parallel, the treatment instrument that is inserted into the second insertion passage is required to be deflectable or steerable as with treatment instrument channels of conventional endoscopes.

The major axis direction of the second insertion passage of the endoscope overtube of the present invention may be different from the major axis direction of the first insertion passage only in the vicinity of the distal end portion. However, if the second insertion passage is curved at a sharp angle in the vicinity of the distal end portion, it is difficult to perform fine manipulation of the treatment instrument inserted into the second insertion passage. Therefore, in consideration of the ease of manipulation of the treatment instrument in the second insertion passage, in particular, the smoothness of the insertion and removal from the distal end, it is preferable that the major axis direction of the second insertion passage forms a helix around the major axis direction of the first insertion passage as a central axis, throughout the second insertion passage. More preferably, it may form a gradual helix.

In terms of the ease of manipulation of the treatment instrument, it is preferable that the helix has a pitch of one turn (360°) or two turns (720°) over the entire length of the second insertion passage. When the pitch is 0.5 turns (180°) or 1.5 turns (540°), the manipulation and the operation of the treatment instrument at the proximal end and the distal end, respectively, are in opposite directions on the right and left sides, and therefore there is the possibility that a manipulation error is induced. When the number of turns is a multiple of 360°, the manipulation and the operation of the treatment instrument at the proximal end and the distal end match, respectively, thus facilitating manipulation. An increase in the number of turns results in an increased angle between the major axis direction of the second insertion passage and the major axis direction of the first insertion passage (the angle of the helix).

The degree of the distance between the center of a surgical field and the distal end of the treatment instrument when the treatment instrument is protruded from the distal end of the overtube is defined according to the major axis direction of the second insertion passage (for example, the angle of the helix), and can be appropriately adjusted according to the extent of the protrusion of the treatment instrument from the second insertion passage.

Usually, a single second insertion passage is provided. Where necessary, two or more second insertion passages may be provided independently of each other. When two or more second insertion passages are present, the major axis directions of the second insertion passages at the distal end of the overtube may be the same as or different from each other. From the viewpoint of maximizing the traction with two forceps, the major axis directions are preferably oriented in directions opposite to each other.

At the distal end of the endoscope overtube of the present invention, the cross section of the wall has an opening for the second insertion passage. The cross section of the wall may be perpendicular to the major axis direction of the first insertion passage, or may have a gradual taper such that the lumen wall side constitutes a distal end (that is, the tip of the overtube). The taper may be appropriately set according to the major axis direction of the second insertion passage (for example, the angle of the helix).

The endoscope is inserted from the proximal end of the first insertion passage, and the endoscope is protruded from the distal end. For example, the treatment instrument is inserted from the proximal end of the second insertion passage, and the treatment instrument is protruded from the distal end thereof. The surfaces of the first and second insertion passages may be provided with coating in order to allow the endoscope to be advanced and retracted smoothly. Preferably, the proximal end of the overtube is provided with a base end portion made of a resin that is harder than the above-described flexible resin in order to facilitate the insertion manipulation of instruments such as the endoscope and the treatment instrument. For example, a grip made of a rigid resin such as an ABS resin may be provided at the base end portion.

According to the present invention, the treatment instrument inserted into the treatment instrument channel of the endoscope inserted into the first insertion passage is protruded toward the center of a surgical field, and therefore a treatment instrument intended for use for incision, coagulation, hemostasis, ablation, morcellation, ligation, cutting and suturing, dissection, and the like is mainly selected.

According to the present invention, although the treatment instrument inserted into the second insertion passage may be deflectable or steerable as long as it can be inserted into the second insertion passage, the treatment instrument preferably may have a relatively small diameter and a simple structure. The treatment instrument in the second insertion passage is protruded in a direction different from the direction of the center of a surgical field, including, for example, a direction away from the center of a surgical field, and therefore a treatment instrument intended to aid the treatment performed with the treatment instrument inserted from the treatment instrument channel of the endoscope described above can be suitably inserted. Examples thereof may include a grasping forceps and a retractor.

According to the present invention, the treatment instrument inserted into the second insertion passage of the overtube can be rotatably moved by turning the endoscope overtube itself without turning the endoscope (with the viewing field of the endoscope held constant), thus enabling increased variety of surgery maneuvers such as an wedge resection of the intestinal lining/mucosa to be realized.

### EXAMPLES

Hereinafter, the present invention will be described in detail with reference to the drawings.

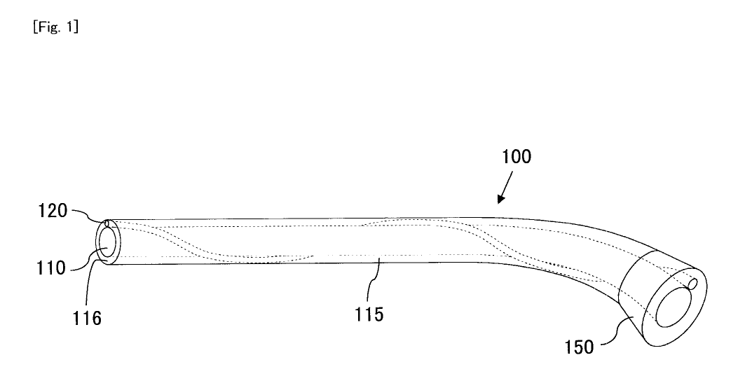

FIG. 1 shows a schematic diagram showing the structure of an endoscope overtube 100 according to the present invention. The endoscope overtube 100 of the present invention includes a first insertion passage 110 into which an endoscope 200 (not shown) is inserted, and a second insertion passage 120 into which a treatment instrument 300 (not shown) is inserted is provided in the form of a helix in a wall 115 constituting the first insertion passage 110. The helix has a pitch of two turns (720°) throughout the entire length of the second insertion passage. The major axis direction of the second insertion passage 120 is different from the major axis direction of the first insertion passage 110 at the distal end of the second insertion passage 120. The proximal end side of the overtube 100 is provided with a base end portion 150 made of a resin that is harder than the flexible resin constituting the wall 115.

As shown in FIG. 1, at the distal end of the endoscope overtube 100 of the present invention, a cross section 116 of the wall 115 has an opening for the second insertion passage 120. In FIG. 1, the cross section 116 of the wall at the distal end is perpendicular to the major axis direction of the first insertion passage 110. However, as described above, the cross section 116 may be a cross section having a taper such that the lumen side of the wall 115 constitutes a distal end (that is, the tip of the overtube 100).

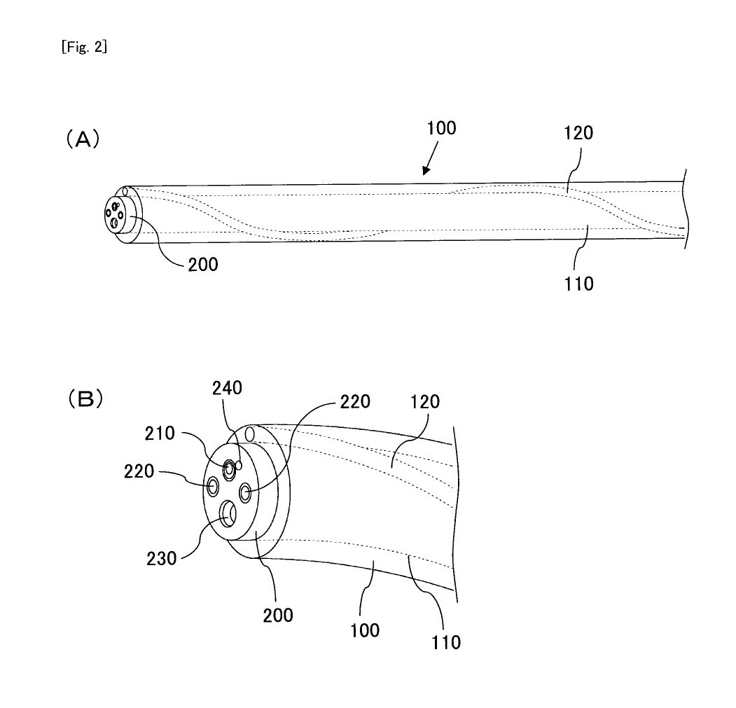

FIG. 2 show a state in which the endoscope 200 is inserted into the first insertion passage 110 of the endoscope overtube 100 shown in FIG. 1. FIG. 2(A) is a perspective view of the distal side of the endoscope overtube 100 when the endoscope 200 is inserted into the first insertion passage 110, and FIG. 2(B) is an enlarged view showing an area in the vicinity of the distal end. Referring to FIG. 2(B), the endoscope 200 is provided, at its tip portion, with an objective lens 210, an illumination light guide 220, a treatment instrument channel 230, and a nozzle 240 for sending out water and air as needed. Preferably, the overtube 100 has a length that can cover the endoscope 200 to the vicinity of the tip portion of the endoscope 200.

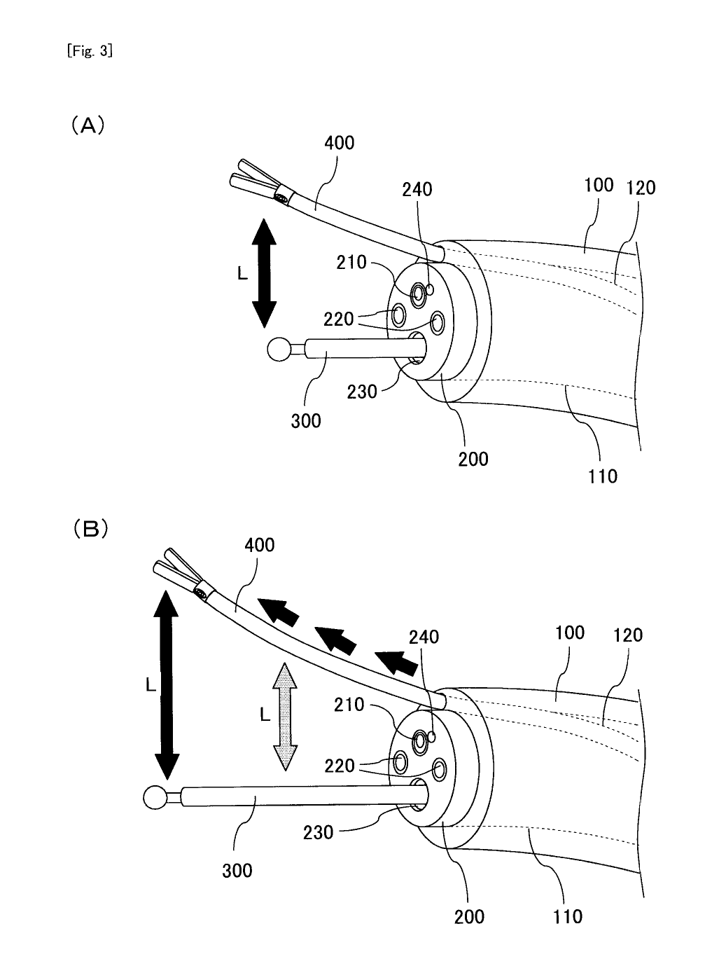

FIG. 3(A) schematically shows a case in which the endoscope 200 is inserted into the first insertion passage 110 of the overtube 100, an electrocautery 300 is inserted into the treatment instrument channel 230 of the endoscope 200, and a grasping forceps 400 is inserted into the second insertion passage 120. The electrocautery 300 extends directly to the center of a surgical field along the viewing axis of the endoscope 200. On the other hand, the forceps 400 inserted into the second insertion passage 120 is protruded along the major axis direction of the second insertion passage 120. Since the major axis direction of the second insertion passage 120 at the distal end is different from the major axis direction of the first insertion passage 110, the grasping forceps 400 is protruded to the outside of the outer circumference of the overtube 100 as indicated by the solid arrows in FIG. 3(B). As can be clearly seen from FIG. 3(B), the distance L between the respective tips of the grasping forceps 400 and the electrocautery 300 (indicated by the double-ended arrows in the drawing) can be adjusted according to the length by which they are protruded.

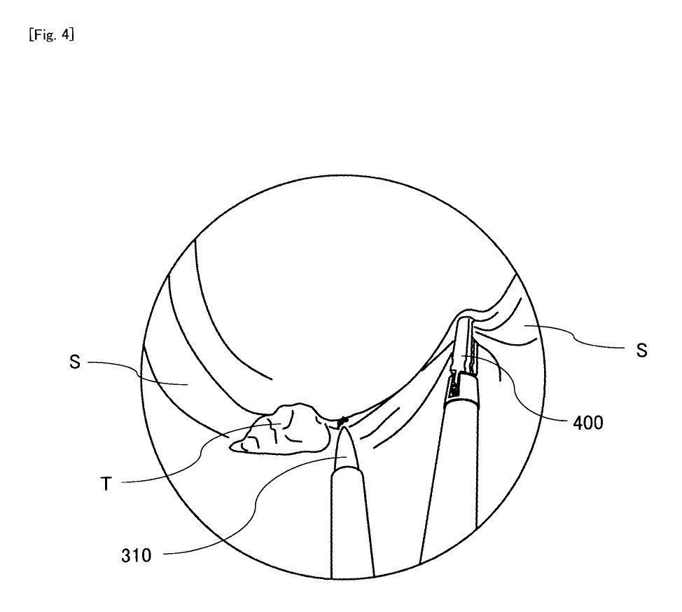

A description will now be given by way of a specific example. FIG. 4 shows a schematic diagram showing a surgical field when endoscopic surgery is performed in a digestive tract by using the endoscope overtube 100. In FIG. 4, an electrocautery 310 having a tip shape different from that in the case of FIG. 3 described above is inserted through the treatment instrument channel 230 of the endoscope 200, and the grasping forceps 400 is inserted through the second insertion passage 120. At the time of removing the tumor T formed on the intestinal surface S with the electrocautery 310, merely bringing only the electrocautery 310 into contact with the portion to be removed will place the intestinal surface S in an unstable state, and therefore it is difficult to freely remove the tumor T. However, as described above, the grasping forceps 400 is protruded to the outside, or in other words, outward from the center of a surgical field, and therefore the intestinal surface S gripped with the grasping forceps 400 can be pulled in a direction obliquely upward and to the right in FIG. 4. Accordingly, it is possible to apply tension on substantially the same plane as the viewing plane, thus providing a tension suitable for removal of the tumor T with the electrocautery 310. The degree of tension can be readily adjusted by adjusting the length of the protrusion of the grasping forceps 400. Furthermore, the distance between the portion treated with the electrocautery 310 and the portion gripped with the grasping forceps 400 can be secured sufficiently, and therefore the visual confirmation of the operation area of the electrocautery 310 can be performed favorably. Thus, when the intestinal surface S is gripped with the grasping forceps 400 and a suitable tension (traction) is applied to the intestinal surface S, the three-dimensional position of the intestinal surface S can be maintained stably, thus greatly facilitating the treatment (removal of the tumor T).

On the other hand, when the grasping forceps 400 inserted into the treatment instrument channel 230 of the endoscope 200 as in conventional technology (not shown), the direction of the tension that can be applied with the grasping forceps 400 is perpendicular to the viewing plane. Specifically, the gripped intestinal surface S is pulled in the front-back direction (that is, forward and rearward) with respect to the viewing plane, and therefore the area that can be visibly confirmed for performing the treatment with the electrocautery 310 is small. With the use of a grasping forceps having a deflectable or steerable structure, the intestinal surface S can be pulled in any direction on substantially the same plane as the viewing plane. However, the structure and the manipulation of the grasping forceps are both complex. In contrast, as described above, in the case of using the endoscope overtube 100, a tension suitable for the treatment can be achieved on the intestinal surface S simply by using a grasping forceps having a simple structure and adjusting the length of the protrusion thereof.

### INDUSTRIAL APPLICABILITY

The endoscope overtube of the present invention does not require an endoscope and a treatment instrument that have a special structure, and can be used together with a commonly used endoscope and a treatment instrument with a simple structure. With the use of the endoscope overtube of the present invention, it is possible to grip and incise a tissue under a suitable tension, and therefore the operability and safety are dramatically improved for technically difficult maneuvers such as EMR, ESD, EAM, EVL, and NOTES. Therefore, the invention is particularly useful for surgical resection of early-staged esophageal cancers, gastric cancers, and colorectal cancers at early stages with EMR and ESD, or advanced intra-abdominal surgery with NOTES.

### DESCRIPTION OF NUMERALS

100 Endoscope overtube
110 First insertion passage
115 Wall
116 Cross section at the distal end
120 Second insertion passage
150 Base end portion
200 Endoscope
210 Objective lens
220 Light guide
230 Treatment instrument channel
240 Nozzle
300, 310 Electrocautery
400 Grasping forceps
L Distance between forceps and electrocautery
S Intestinal surface
T Tumor

1. An endoscope overtube comprising a first insertion passage into which an endoscope is inserted,
wherein a wall constituting the first insertion passage comprises a second insertion passage into which a treatment instrument is inserted, and a major axis direction of the second insertion passage is different from a major axis direction of the first insertion passage at a distal end of the second insertion passage

2. The endoscope overtube according to claim 1, wherein the major axis direction of the second insertion passage forms a helix around the major axis direction of the first insertion passage as a central axis, throughout the second insertion passage.

3. The endoscope overtube according to claim 2, wherein the helix has a pitch of one turn (360°) or two turns (720°) throughout the second insertion passage.

4. The endoscope overtube according to claim 1, wherein the second insertion passage is a lumen that is independent of the first insertion passage.

5. A method for using an endoscope overtube, comprising the step of turning an endoscope overtube around an endoscope.

6. The endoscope overtube according to claim 2, wherein the second insertion passage is a lumen that is independent of the first insertion passage.

7. The endoscope overtube according to claim 3, wherein the second insertion passage is a lumen that is independent of the first insertion passage.
